(19) United States
(12) Patent Application Publication
(10) Pub. No.: US 2021/0186342 A1
(43) Pub. Date: Jun. 24, 2021

(54) INTELLIGENT HEAD PROTECTIVE EQUIPMENT

(71) Applicant: Cipher Skin, Denver, CO (US)

(72) Inventor: Phillip Bogdanovich, Denver, CO (US)

(73) Assignee: Cipher Skin, Denver, CO (US)

(21) Appl. No.: 17/133,955

(22) Filed: Dec. 24, 2020

Related U.S. Application Data
(60) Provisional application No. 62/953,334, filed on Dec. 24, 2019.

(51) Int. Cl. A61B 5/0205; G16H 40/67; A61B 5/00; A61B 5/145

(52) U.S. Cl. CPC A61B 5/02055; G16H 40/67; A61B 5/6803; A61B 5/742; A61B 5/7405; G06F 1/163; A61B 5/14542; A61B 5/14517; A61B 5/14532; A61B 5/0022; A61B 5/7282; A61B 5/7455

(57) ABSTRACT

An intelligent wearable device is disclosed. The intelligent wearable device is configured to monitor and obtain biological data associated with a user wearing the device and environmental data associated with an environment that the user is located in through the use of a plurality of sensors of the wearable device. Once the biological and environmental data are obtained, the intelligent wearable device may compare the data to a threshold value that is associated with the safety of the user. If the comparison indicates that the data is outside the threshold value, the intelligent wearable device may output a signal indicating that the user is experiencing an event. The intelligent wearable device may also generate feedback indicating an action to perform in response to the event. The intelligent wearable device may notify devices associated with other users, who may be able to assist the user experiencing the event.

### CROSS-REFERENCE TO RELATED APPLICATIONS

This application claims priority to U.S. Patent Application No. 62/953,334, filed on Dec. 24, 2019, which is incorporated by reference as if fully set forth herein.

### FIELD OF THE INVENTION

The present application relates to wearable technologies, safety technologies, sensor technologies, health-related technologies, environment analysis technologies, data aggregation and analysis technologies, database technologies, and computing technologies, and more particularly, to a system and method for providing and/or utilizing an intelligent wearable device, such as an intelligent head protective equipment.

### BACKGROUND

In today's society, various devices exist for protecting users in various types of environments. For example, Industrial Personal Protective Equipment (PPE) is often worn in places where the environment needs to be continuously monitored for changes that could be detrimental to the wearer of the PPE. One such type of equipment is Head PPE (H-PPE), which is a common and often required protective equipment. Such equipment is often worn by users working in dangerous environments, such as, but not limited to, oil refineries, ships, factories, mines, and industrial plants. In these types of environments rapid changes in temperature as well as other factors are often experienced by users. These types of rapid changes can alter the relative safety of the user and can include risks, such as, but not limited to, chemical exposure, hypothermia and hyperthermia.

Notably, users often ignore feelings and signs associated with negative changes in personal safety. For instance, users may ignore, or, in certain circumstances, may not even perceive feeling hot and disoriented immediately prior to an acute hyperthermic reaction, such as a heat stroke. Additionally, negative changes to worker safety have a massive impact on worker effectiveness, general productivity, and company cash flow. When workers are injured, sections of jobsites and the incident location often have to be taken offline, which ultimately cost the company money and decrease worker productivity. As a result, ensuring safety compliance effectively involves providing supervisors with a high level of oversight of subordinate health. Currently, such oversight is not possible especially at scale where one supervisor has reliable oversite of multiple subordinates.

Notably, heat-related injuries cost companies significant amounts of money each year in terms of downtime, hospital costs and education. It is estimated that thermal injuries are thirteen times more likely to be experienced in industrial environments. Such industrial environments tend to be associated with industries that have the least efficient monitoring methods. While attempts have been made to increase wearer safety in adverse conditions, such attempts are still not effective enough. For instance, currently technologies provide for the integration of fire retardant into oil field and fire fighter garments. Notably, adding material to prevent or limit life threatening injuries is a reactive measure and will only reduce injury severity on occasions where injuries of some sort are imminent. As a result, providing the capability to monitor for impending danger and changes that indicate that workers are or are about to be in danger would be very helpful.

Based on the foregoing, current technologies and processes may be modified and improved so as to provide enhanced functionality and features for users and systems to effectively monitor an individual's safety, detect health conditions or other types of events, detect physiological statuses and events, and determine actions for dealing with potential health concerns and events. Such enhancements and improvements may provide for improved user satisfaction, increased safety, increased reliability, increased accuracy, increased efficiencies, increased access to meaningful data, substantially-improved decision-making abilities, substantially reduced costs for businesses and individuals, and increased ease-of-use for users.

### SUMMARY

An intelligent wearable device, and a system and methods for providing and utilizing an intelligent wearable device are disclosed. In particular, intelligent wearable device may be an intelligent head protective equipment that may be worn on the head of a user, and may be utilized to monitor the health and other metrics associated with the user and/or the user's environment. In certain embodiments, the intelligent wearable device may be modified or adapted to fit on other body parts of the user. Notably, the system and methods may include utilizing sensors of the intelligent wearable device to monitor health data associated with the user, environmental data associated with an environment associated with the user, any other data, or a combination thereof. The various types of data may be monitored by utilizing various sensors of the intelligent wearable device, which may include, but are not limited to, temperature sensors, pressure sensors, motion sensors, light sensors, oxygen sensors, heart rate sensors, touch sensors, proximity sensors, gas sensors, acoustic sensors, chemical sensors, acceleration sensors, humidity sensors, moisture sensors, presence sensors, force sensors, any type of sensors, or a combination thereof. The various sensors of the intelligent wearable device may be configured to monitor the health-related data and the environmental data, and may provide the data to one or more processors of the intelligent wearable device for processing via one or more signals.

Once the health-related data and the environmental data are received by the processor(s) of the intelligent wearable device, the system and methods may include comparing the data to a threshold range of values or a threshold value that may be associated with the health and/or safety of the user. If the comparison indicates that the data lies within the threshold range of values associated with the health and/or safety of the user (or not beyond a threshold value), the system and methods can include continuing to monitor and compare the data. If, however the comparison indicates that the data lies outside the threshold range of values (or beyond a threshold value) associated with the health and/or safety of the user, the system and methods may include generating a signal that indicates that the user is experiencing some type of event. For example, the event may be that the user is experiencing an extremely high body temperature and/or environmental temperature, that the user is experiencing a heart rate problem, that the user is experiencing an oxygen problem, that the user is experiencing any type of health-related issue, or a combination thereof. The system and methods may include utilizing the generated signal to output a notification using the intelligent wearable device advising of the event and/or one or more actions to perform in response to the detection of the event. In certain embodiments, the intelligent wearable device may transmit the signal to other devices associated with other users to notify such users of the event and/or actions to perform to assist the first user in dealing with or responding to the event.

To that end, in one embodiment according to the present disclosure, an intelligent wearable device is disclosed. In particular, the intelligent wearable device may comprise intelligent head protective equipment, which may be worn on the head of a user. In certain embodiments, the intelligent wearable device may be configured to be modified to fit on other body parts of the user. The intelligent wearable device may include a helmet, one or more sensors coupled to the helmet, a suspension system configured to reside within the helmet, any number of memories, any number of processors, any number of transceivers, any number of speakers, any number of microphones, any number of user interfaces, a wireless chip for communicating with one or more networks, any number of other components, or a combination thereof. The one or more memories may store instructions and the one or more processors may execute the instructions to perform operations conducted by the intelligent wearable device. In certain embodiments, intelligent wearable device may perform an operation that includes receiving a first signal from one or more sensor(s). The first signal may include health-related data associated with a first user wearing the wearable device and/or environmental data associated with an environment that the first user is located in. Additionally, the intelligent wearable device may perform an operation that includes comparing the health-related data associated with the first user, the environmental data, or a combination thereof, to a threshold range of values (or a threshold value) associated with safety of the first user. Furthermore, the intelligent wearable device may perform an operation that includes generating a second signal indicating that the first user is experiencing an event if the comparing indicates that the health-related data, the environmental data, or both, are outside the threshold range of values (or beyond a threshold value). In certain embodiments, the intelligent wearable device may output a notification providing information relating to the event and/or an action to perform in response to the occurrence of the event. In further embodiments, the intelligent wearable device may transmit the second signal to another device associated with a second user to notify the second user of the event and/or actions to perform with respect to the event.

In another embodiment, a method for utilizing an intelligent wearable device, such as intelligent head protective equipment is disclosed. The method may include utilizing a memory that stores instructions, and a processor that executes the instructions to perform the various functions of the method. In particular, the method may include positioning the intelligent wearable device on a first user, such as on the head of the first user. The method may then include activating the intelligent wearable device. Once the intelligent wearable device is activated, the method may include receiving a first signal(s) from one or more sensors of the intelligent wearable device that includes health-related data associated with the first user, environmental data associated with an environment associated with the first user, or a combination thereof. The method may then include comparing the health-related data, the environmental data, or a combination thereof, to a threshold range of values (or a threshold value) associated with the safety of the first user. The method may include determining if the comparing indicates that the health-related data, the environmental data, or both, are outside the threshold range of values (or beyond the threshold value). If the health-related data, the environmental data, or both, are not outside the threshold range of values (or the threshold value), the method may include continuing to receive signals from the one or more sensors. If, however, the health-related data, the environmental data, or both, are outside the threshold range of values (or the threshold value), the method may include generating a second signal indicating that the first user is experiencing an event. In certain embodiments, the method may include outputting, by utilizing the second signal, a notification providing information relating to the event and/or to an action to perform in response to the event. In certain embodiments, the method may also include transmitting the second signal to a device of a different user to notify the different user of the event and/or an action to perform with respect to the event.

According to yet another embodiment, a computer-readable device having instructions for utilizing an intelligent wearable device is provided. The computer instructions, which when loaded and executed by a processor, may cause the processor to perform operations including: receiving a first signal from a sensor of a wearable device, wherein the first signal includes health-related data associated with a first user of the wearable device and environmental data associated with an environment associated with the first user; comparing, by utilizing instructions executed by a processor of the wearable device, the health-related data associated with the first user, the environmental data, or a combination thereof, to a threshold range of values (or a threshold value) associated with safety of the first user; and determining, if the comparing indicates that the health-related data, the environmental data, or both, are outside the threshold range of values (or the threshold value), that the first user is experiencing an event.

These and other features of the systems and methods for providing and/or utilizing an intelligent wearable device are described in the following detailed description, drawings, and appended claims.

### BRIEF DESCRIPTION OF THE DRAWINGS

FIG. 1 is a schematic diagram of a system for utilizing and interacting with intelligent head protective equipment according to an embodiment of the present disclosure.

FIG. 2 is a schematic diagram illustrating various features and components of the intelligent head protective equipment of FIG. 1 according to an embodiment of the present disclosure.

FIG. 3 is a schematic diagram illustrating additional features and components of the intelligent head protective equipment of FIG. 1 according to an embodiment of the present disclosure.

FIG. 4 is a flow diagram illustrating a sample method for utilizing intelligent head protective equipment according to an embodiment of the present disclosure.

FIG. 5 is a schematic diagram of a machine in the form of a computer system within which a set of instructions, when executed, may cause the machine to perform any one or more of the methodologies or operations of the systems and methods for providing and utilizing intelligent head protective equipment.

### DETAILED DESCRIPTION OF THE INVENTION

A system 100 and methods for providing and utilizing an intelligent wearable device are disclosed. In particular, intelligent wearable device (e.g. intelligent wearable devices 300, 400) may be an intelligent head protective equipment that may be worn on the head or other body part of a user, and may be utilized to monitor the health and other metrics associated with the user and/or the user's environment. Notably, the system 100 and methods may include utilizing sensors of the intelligent wearable device to monitor biological and/or health data associated with the user, environmental data associated with an environment associated with the user, any other data, or a combination thereof. The various types of data may be monitored by utilizing various sensors of the intelligent wearable device. Such sensors may include, but are not limited to, temperature sensors, pressure sensors, motion sensors, light sensors, oxygen sensors, heart rate sensors, touch sensors, proximity sensors, gas sensors, acoustic sensors, chemical sensors, acceleration sensors, humidity sensors, moisture sensors, presence sensors, force sensors, any type of sensors, or a combination thereof. In certain embodiments, the various sensors of the intelligent wearable device may be configured to monitor the health-related data and the environmental data, and may provide the data to one or more processors of the intelligent wearable device for processing via one or more signals.

Once the biological data, health-related data and/or the environmental data are received by the processor(s) of the intelligent wearable device, the system 100 and methods may include comparing the data to a threshold range of values or to a threshold value that may be associated with the health and/or safety of the user (or safety in general). If the comparison indicates that the data lies within the threshold range of values associated with the health and/or safety of the user (or not beyond a threshold value), the system and methods can include continuing to monitor and compare the data. If, however the comparison indicates that the data lies outside the threshold range of values (or beyond a threshold value) associated with the health and/or safety of the user, the system and methods may include generating a signal that indicates that the user is experiencing some type of event. For example, the event may be that the user is experiencing an extremely high body temperature and/or environmental temperature, that the user is experiencing a heart rate problem, that the user is experiencing an oxygen problem, that the user is experiencing any type of health-related issue, or a combination thereof. The system and methods may include utilizing the generated signal to output a notification using the intelligent wearable device advising of the event and/or one or more actions to perform in response to the detection of the event. In certain embodiments, the intelligent wearable device may transmit the signal to other devices associated with other users to notify such users of the event and/or actions to perform to assist the first user in dealing with or responding to the event.

In certain exemplary embodiments, the intelligent wearable device may be utilized to measure and track the thermal condition of a user in conjunction with a host of other metrics. The intelligent wearable device may utilize a plurality of sensors (e.g. temperature sensors) to compare the skin temperature (e.g. from the forehead of the user) to the temperature inside of the helmet of the intelligent wearable device and map the correlation to the user's oral temperature, which may be measured by another sensor or even a thermometer. Notably, the components providing the operative functionality of the intelligent wearable device may either be retrofitted to the interior of an existing helmet device or integrated directly into a helmet prior to being provided to a consumer. Using the functionality provided by the system 100 and methods, the intelligent wearable device may transmit information associated with the user to a supervisor or other third-party via an application, digital dashboard, or alert. In certain embodiments, the user himself may be notified via the intelligent wearable device itself when the user's temperature (or other measured metric) either falls below or climbs above presets safe values. Such notifications may include, but are not limited to, haptic feedback, audio signals (e.g. audio alerts), and/or periphery visual signals (e.g. lights).

Notably, the system 100 and methods facilitate the monitoring and evaluation of the general health and safety of a user, especially in locations which may be associated with a lack of safety. The system 100 and methods also facilitate providing feedback to the user as it relates the user's personal safety with regard to the user's body temperature and general state (e.g. ECG, SPO2, etc.). In certain embodiments, the system 100 and methods take advantage of the modular nature of the sensors described in the present disclosure by incorporating sensors into a flexible primary sensor (e.g. such as by incorporating the sub-sensors and primary sensor via the sensor strip 220). In certain embodiments, the system 100 and methods may allow the sub-sensors to be swapped out of the flexible primary sensor so that the functionality of the system 100 and methods may be adapted to desired environments, use-case scenarios, and/or requirements. In further embodiments, the system 100 and methods also allow for the long-term health of a user to be monitored and for the correlation of changes in health based on various types of environmental exposure.

Still further, the system 100 and methods monitors not only the physical environment but also biological signals and how environmental information and inputs impact biological signals and vice versa. The system 100 and methods facilitate monitoring and tracking sensory inputs over time and thus are capable of generating data taken over some period of time that can then be mined for correlation between environmental factors and biological changes to the user. The intelligent wearable devices and accompanying sensors of the system 100 and methods are modular in nature and comprise the first design that allows for the retrofitting of smart capabilities into existing H-PPE hardware.

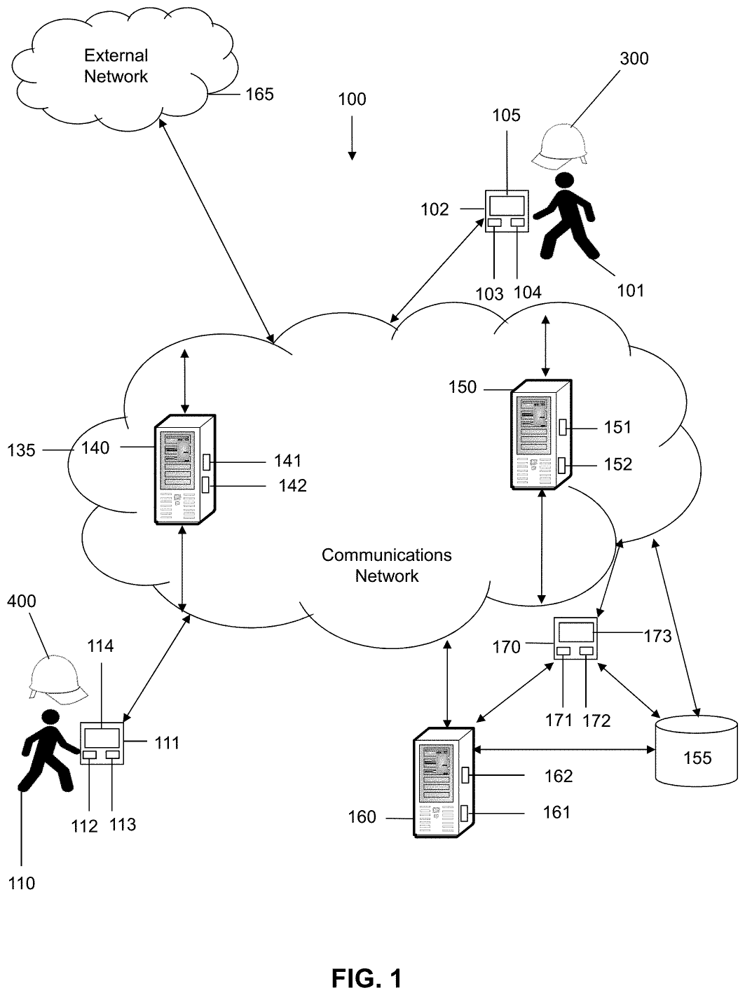

As shown in FIGS. 1-5, a system 100 and method for providing and utilizing an intelligent wearable device, such as intelligent head protective equipment are disclosed. The system 100 may be configured to support, but is not limited to supporting, monitoring applications and services, sensor-based applications and services, wearable device applications and services, health monitoring applications and services, communication applications and services, alert applications and services, data and content services, data aggregation applications and services, big data technologies, health analysis technologies, data synthesis applications and services, data analysis applications and services, computing applications and services, cloud computing services, internet services, satellite services, telephone services, software as a service (SaaS) applications, mobile applications and services, and any other computing applications and services. The system may include a first user 101, who may utilize a first user device 102 to access data, content, and applications, or to perform a variety of other tasks and functions. As an example, the first user 101 may utilize first user device 102 to access an application (e.g. a browser or a mobile application) executing on the first user device 102 that may be utilized to access web pages, data, and content associated with the system 100. In certain embodiments, the first user 101 may be a user that is a worker at an industrial plant, oil refinery, ship, factory, and/or any other location that may potentially expose the first user 101 to potentially dangerous conditions.

The first user device 102 utilized by the first user 101 may include a memory 103 that includes instructions, and a processor 104 that executes the instructions from the memory 103 to perform the various operations that are performed by the first user device 102. In certain embodiments, the processor 104 may be hardware, software, or a combination thereof. The first user device 102 may also include an interface 105 (e.g. screen, monitor, graphical user interface, audio device interface, etc.) that may enable the first user 101 to interact with various applications executing on the first user device 102, to interact with various applications executing within the system 100, and to interact with the system 100 itself. In certain embodiments, the first user device 102 may be a computer, a laptop, a tablet device, a phablet, a server, a mobile device, a smartphone, a smart watch, and/or any other type of computing device. Illustratively, the first user device 102 is shown as a mobile device in FIG. 1. The first user device 102 may also include a global positioning system (GPS), which may include a GPS receiver and any other necessary components for enabling GPS functionality, accelerometers, gyroscopes, sensors, and any other componentry suitable for a mobile device. In certain embodiments, the first user device 102 may be configured to include any number of sensors, such as, but not limited to, temperature sensors, pressure sensors, motion sensors, light sensors, oxygen sensors, heart rate sensors, touch sensors, proximity sensors, gas sensors, acoustic sensors, chemical sensors, acceleration sensors, humidity sensors, moisture sensors, presence sensors, force sensors, any type of sensors, or a combination thereof.

In addition to the first user 101, the system 100 may include a second user 110, who may utilize a second user device 111 to access data, content, and applications, or to perform a variety of other tasks and functions. As with the first user 101, the second user 110 may be a user that is a worker at an industrial plant, oil refinery, ship, factory, and/or any other location that may potentially expose the first user 101 to potentially dangerous conditions. However, in certain embodiments, the second user 110 may be a supervisor of the first user 101, a physician, a first responder, an emergency personnel, a nurse, any type of health professional, any type of safety personnel, or any combination thereof. Much like the first user 101, the second user 110 may utilize second user device 111 to access an application (e.g. a browser or a mobile application) executing on the second user device 111 that may be utilized to access web pages, data, and content associated with the system 100. The second user device 111 may include a memory 112 that includes instructions, and a processor 113 that executes the instructions from the memory 112 to perform the various operations that are performed by the second user device 111. In certain embodiments, the processor 113 may be hardware, software, or a combination thereof. The second user device 111 may also include an interface 114 (e.g. a screen, a monitor, a graphical user interface, etc.) that may enable the second user 110 to interact with various applications executing on the second user device 111, to interact with various applications executing in the system 100, and to interact with the system 100. In certain embodiments, the second user device 111 may be a computer, a laptop, a tablet device, a phablet, a server, a mobile device, a smartphone, a smart watch, and/or any other type of computing device. Illustratively, the second user device 111 may be a computing device in FIG. 1. The second user device 111 may also include any of the componentry described for first user device 102.

In certain embodiments, the first user device 102 and the second user device 111 may have any number of software applications and/or application services stored and/or accessible thereon. For example, the first and second user devices 102, 111 may include applications for determining and analyzing health conditions, applications for determining and analyzing the physiological status of a user, applications for generating alerts, applications for analyzing and interpreting sensor data, artificial intelligence applications, machine learning applications, big data applications, applications for analyzing data, applications for integrating data, cloud-based applications, search engine applications, natural language processing applications, database applications, algorithmic applications, phone-based applications, product-ordering applications, business applications, e-commerce applications, media streaming applications, content-based applications, database applications, gaming applications, internet-based applications, browser applications, mobile applications, service-based applications, productivity applications, video applications, music applications, social media applications, presentation applications, any other type of applications, any types of application services, or a combination thereof. In certain embodiments, the software applications and services may include one or more graphical user interfaces so as to enable the first and second users 101, 110 to readily interact with the software applications.

The software applications and services may also be utilized by the first and second users 101, 110 to interact with any device in the system 100, any network in the system 100, or any combination thereof. For example, the software applications executing on the first and second user devices 102, 111 may be applications for receiving data, applications for storing data, applications for determining health conditions, applications for determining how to respond to a health condition, applications for determining a physiological status of a user, applications for determining how to respond to an environmental condition (e.g. an environmental condition that may affect the first user 101), applications for receiving demographic and preference information, applications for transforming data, applications for executing mathematical algorithms, applications for generating and transmitting electronic messages, applications for generating and transmitting various types of content, any other type of applications, or a combination thereof. In certain embodiments, the first and second user devices 102, 111 may include associated telephone numbers, internet protocol addresses, device identities, or any other identifiers to uniquely identify the first and second user devices 102, 111 and/or the first and second users 101, 110. In certain embodiments, location information corresponding to the first and second user devices 102, 111 may be obtained based on the internet protocol addresses, by receiving a signal from the first and second user devices 102, 111, or based on profile information corresponding to the first and second user devices 102, 111.

The system 100 may also include a communications network 135. The communications network 135 of the system 100 may be configured to link each of the devices in the system 100 to one another. For example, the communications network 135 may be utilized by the first user device 102 to connect with other devices within or outside communications network 135. Additionally, the communications network 135 may be configured to transmit, generate, and receive any information and data traversing the system 100. In certain embodiments, the communications network 135 may include any number of servers, databases, or other componentry, and may be controlled by a service provider. The communications network 135 may also include and be connected to a cloud-computing network, a phone network, a wireless network, an Ethernet network, a satellite network, a broadband network, a cellular network, a private network, a cable network, the Internet, an internet protocol network, a content distribution network, any network, or any combination thereof. Illustratively, server 140 and server 150 are shown as being included within communications network 135.

Notably, the functionality of the system 100 may be supported and executed by using any combination of the servers 140, 150, and 160. The servers 140, and 150 may reside in communications network 135, however, in certain embodiments, the servers 140, 150 may reside outside communications network 135. The servers 140, and 150 may be utilized to perform the various operations and functions provided by the system 100, such as those requested by applications executing on the first and second user devices 102, 111. In certain embodiments, the server 140 may include a memory 141 that includes instructions, and a processor 142 that executes the instructions from the memory 141 to perform various operations that are performed by the server 140. The processor 142 may be hardware, software, or a combination thereof. Similarly, the server 150 may include a memory 151 that includes instructions, and a processor 152 that executes the instructions from the memory 151 to perform the various operations that are performed by the server 150. In certain embodiments, the servers 140, 150, and 160 may be network servers, routers, gateways, switches, media distribution hubs, signal transfer points, service control points, service switching points, firewalls, routers, edge devices, nodes, computers, mobile devices, or any other suitable computing device, or any combination thereof. In certain embodiments, the servers 140, 150 may be communicatively linked to the communications network 135, any network, any device in the system 100, or any combination thereof.

The database 155 of the system 100 may be utilized to store and relay information that traverses the system 100, cache information and/or content that traverses the system 100, store data about each of the devices in the system 100, and perform any other typical functions of a database. In certain embodiments, the database 155 may store the output from any operation performed by the system 100, operations performed and/or outputted by the intelligent wearable devices 300, 400, operations performed and/or outputted by the sensors 225, 230, 235, 240 operations performed and/or outputted by any component, program, process, device, network of the system 100, or any combination thereof. For example, the database 155 may store data from data sources, such as, but not limited to, sensors 225, 230, 235, which may measure sensor data associated with the first user 101 and/or an environment that the first user 101 is located in. In certain embodiments, the database 155 may be connected to or reside within the communications network 135, any other network, or a combination thereof. In certain embodiments, the database 155 may serve as a central repository for any information associated with any of the devices and information associated with the system 100. Furthermore, the database 155 may include a processor and memory or be connected to a processor and memory to perform the various operations associated with the database 155. In certain embodiments, the database 155 may be connected to the servers 140, 150, 160, the first user device 102, the second user device 111, intelligent wearable devices 300, 400, any devices in the system 100, any other device, any network, or any combination thereof.

The database 155 may also store information obtained from the system 100, store information associated with the first and second users 101, 110, store location information for the first and second user devices 102, 111 and/or first and second users 101, 110, store user profiles associated with the first and second users 101, 110, store device profiles associated with any device in the system 100 (e.g. the intelligent wearable devices 300, 400), store communications traversing the system 100, store user preferences, store demographic information for the first and second users 101, 110, store information associated with any device or signal in the system 100, store information relating to usage of applications accessed by the first and second user devices 102, 111, store any information obtained from any of the networks in the system 100, store historical data associated with the first and second users 101, 110, store device characteristics, store information relating to any devices associated with the first and second users 101, 110, or any combination thereof. The database 155 may store algorithms for analyzing sensor data obtained from the sensors 225, 230, 235, 240, algorithms for determining events, such as health conditions and/or physiological status, algorithms for determining activities that the users are suited for, algorithms conducting artificial intelligence and/or machine learning, algorithms for comparing sensor data to baseline and/or threshold values, any other algorithms for performing any other calculations and/or operations in the system 100, or any combination thereof. The database 155 may also be configured to store information relating to detected events, actions to perform in response to the detected events, information indicating whether one or more of the actions have been performed, information indicating which environment associated with what types of events and how the events typically occur, any other information provided by the system 100 and/or method 400, or any combination thereof. In certain embodiments, the database 155 may be configured to store any information generated and/or processed by the system 100, store any of the information disclosed for any of the operations and functions disclosed for the system 100 herewith, store any information traversing the system 100, or any combination thereof. Furthermore, the database 155 may be configured to process queries sent to it by any device in the system 100.

The system 100 may also include a software application, which may be configured to perform and support the operative functions of the system 100. In certain embodiments, the application may be a website, a mobile application, a software application, or a combination thereof, which may be made accessible to users utilizing one or more computing devices, such as first user device 102 and second user device 111. The application of the system 100 may be accessible via an internet connection established with a browser program executing on the first or second user devices 102, 111, a mobile application executing on the first or second user devices 102, 111, or through other suitable means. Additionally, the application may allow users and computing devices to create accounts with the application and sign-in to the created accounts with authenticating username and password log-in combinations. The application may include a custom graphical user interface that the first user 101 or second user 110 may interact with by utilizing a web browser executing on the first user device 102 or second user device 111. In certain embodiments, the software application may execute directly as an installed program on the first and/or second user devices 102, 111. In certain embodiments, some or all of the software application may execute on the intelligent wearable devices 300, 400.

The software application may include multiple programs and/or functions that execute within the software application and/or are accessible by the software application. For example, the software application may include an application that generates web content, pages, and/or data that may be accessible to the first and/or second user devices 102, 111, the intelligent wearable devices 300, 400, the database 155, the external network 165, any type of program, any device and/or component of the system 100, or any combination thereof. The application that generates web content and pages may be configured to generate a graphical user interface and/or other types of interfaces for the software application that is accessible and viewable by the first and second users 101, 110 when the software application is loaded and executed on the first and/or second computing devices 102, 111. The graphical user interface for the software application may display content associated with health conditions, sensor data measured by the sensors 225, 230, 235, 240, physical measurements of users, environmental data measurements, correlations of environmental data with health conditions, detected health events or other events, actions to perform in response to detected events, any other type of information, or any combination thereof. Additionally, the graphical user interface may display functionality provided by the software application that enables the first and/or second user 101, 110 and/or the first user device and/or second user device 111 to input parameters and requirements for the various process conducted by the system 100.

The system 100 may also include an external network 165. The external network 165 of the system 100 may be configured to link each of the devices in the system 100 to one another. For example, the external network 165 may be utilized by the first user device 102, the second user device 111, the intelligent wearable device 300, and/or the intelligent wearable device 400 to connect with other devices within or outside communications network 135. Additionally, the external network 165 may be configured to transmit, generate, and receive any information and data traversing the system 100. In certain embodiments, the external network 165 may include any number of servers, databases, or other componentry, and may be controlled by a service provider. The external network 165 may also include and be connected to a cloud-computing network, a phone network, a wireless network, an Ethernet network, a satellite network, a broadband network, a cellular network, a private network, a cable network, the Internet, an internet protocol network, a content distribution network, any network, or any combination thereof. In certain embodiments, the external network 165 may be outside the system 100 and may be configured to perform various functionality provided by the system 100, such as if the system 100 is overloaded and/or needs additional processing resources. In certain embodiments, the external network 165 may be configured to perform some or all of the operations conducted by the intelligent wearable devices 300, 400.

The system 100 may also include one or more intelligent wearable devices 300, 400. In certain embodiments, the intelligent wearable devices 300, 400 may take the form of a hard hat, a helmet, a shoe, a jacket, pants, armor, a belt, a visor, an undergarment, a sock, a tie, a watch, a necklace, any type of item of clothing, any device that can be worn, anything that can be worn, or any combination thereof. In a preferred embodiment, the intelligent wearable devices 300, 400 may take the form of a hard hat, which may include a suspension system 200. Notably, the intelligent wearable device 300 may include any of the componentry of intelligent wearable device 400 and vice versa. The intelligent wearable device 300 (and 400) may include one or more of the following components: a helmet portion 305, a bill portion 310, an interface 315, a microphone 320, a speaker 325, a haptic component 330 (i.e. any type of device capable of haptic functionality), a transceiver 335, a suspension system 200, a tightening device 205, straps 210, a processor 215, a light device 217 (e.g. LED or other light device), a sensor strip 220, sensors 225, 230, 235, 240, one or more memories, one or more headphones, one or more short range and/or long range wireless protocol-enabled chips, one or more radios, any number of batteries or other power sources, any other desired components, or any combination thereof. In certain embodiments, the components of the intelligent wearable devices 300 may be directly connected to each other, however, in certain embodiments, the components may commutatively link to each other via short-range wireless connections (e.g. Bluetooth, Zigbee, Z-wave, etc.). In certain embodiments, one or more of the components of the intelligent wearable device 300 may reside on a printed circuit board, which may link the components to each other. The helmet portion 305 of the intelligent wearable device 300 may include an opening that may be configured to rest upon the head of a user when secured to the user, and the bill portion 310 may be configured to provide shade to the face of the user and/or protection to the user in the event that debris falls in proximity to the user's face.

The interface 315 of the intelligent wearable device 300 may be an interface that may be configured to display data, alerts, media content, any type of content, or a combination thereof. For example, the interface 315 may be configured to display or otherwise output sensor data, an identification of an event being experienced by the users wearing the intelligent wearable device 300, a visual alert relating to the event, an auditory alert relating to the event, information indicative of an action to perform in response to the event, any type of information, or a combination thereof. In certain embodiments, the interface 315 may be positioned on the side of the helmet portion 305, however, the interface 315 may be positioned on any desired location on the intelligent wearable device 300. In certain embodiments, the interface 315 may reside on a device separate from the intelligent wearable device 300, but separate device including the interface 315 may be secured to the helmet portion 305 and/or bill portion 310 of the intelligent wearable device 300, such as by utilizing a fastening or attachment device.

In certain embodiments, the intelligent wearable device 300 may include a microphone 320, which may include any of the componentry of any microphone and may be configured to receive and process audio signals. For example, the microphone 320 may be configured to pick up audio signals generated by a user himself and/or any audio signals occurring in an environment that the user is located in. The microphone 320 may also be configured to receive and process audio signals outputted from other components of the intelligent wearable device 300, and any other device and/or user of the system 100. In certain embodiments, the microphone 320 may be positioned on any desired location of the intelligent wearable device 300 and may even be configured to be a separate device that is attachable to any portion of the intelligent wearable device 300. The intelligent wearable device 300 may also include a speaker 325, which may be configured to output audio signals received by the microphone 320, received by any other device of the system 100, received by any user of the system 100, or any combination thereof. In certain embodiments, the audio signals may be audio alerts that indicate an event experienced by a user, an action to perform to assist the user with the event, any other information, or a combination thereof. As with the microphone, the speaker 320 may be positioned on any desired location of the intelligent wearable device 300 and may even be configured to a be separate device that is attachable to any portion of the intelligent wearable device 300.

The intelligent wearable device 300 may also include a haptic component 330. The haptic component 330 may be configured to emit vibrations in random patterns, sequenced patterns, or a combination thereof. The haptic component 330 may be configured to receive signals from other components of the intelligent wearable device 300, other devices of the system 100, or a combination thereof, which may cause the haptic component 330 to vibrate in a particular manner. For example, if the user is experiencing an event, a component of the intelligent wearable device 300 may provide a signal to the haptic component 330 to output a sequence of five vibrations. As another example, if the intelligent wearable device 300 is trying to notify a user of an action to perform in response to a detected event, a signal may be provided to the haptic component 330 to alternate long and short vibrations according to a specific pattern. For example, a specific pattern of the alternate long and short vibrations may indicate that the user needs to run away from the current location that the user is located in to avoid continued exposure to something occurring in the environment that the user is currently located in.

In certain embodiments, the intelligent wearable device 300 may include a transceiver 335. The transceiver 335 may include any of the functionality of a traditional transceiver. The transceiver 335 may be configured to transmit signals generated by the components of the intelligent wearable device 300 to other devices and networks of the system 100, receive signals generated by any of the devices of the system 100, receive signals generated by the components of the intelligent wearable device 300, or a combination thereof. In certain embodiments, the transceiver 335 may enable the intelligent wearable device to communicate information gathered by the components of the intelligent wearable device to other devices of the system 100 for further processing. In certain embodiments, the transceiver 335 may enable the intelligent wearable device 300 to communicatively coupled to any device in the system 100, the communications network 135, the external network 165, or a combination thereof.

In certain embodiments, the intelligent wearable device 300 may be configured to couple to a suspension system 200. The suspension system 200 may be component that may be configured to absorb shocks and blows to the head of a user when the suspension system 200 is secured to the underside of the helmet portion 305 of the intelligent wearable device 300. The suspension system 200 may be configured to provide a gap between the helmet portion 305 and the head of the user, and may redirect impact forces affecting the helmet portion 305 away from the user's head, spine, and/or other body parts. In certain embodiments, the suspension system 200 may include any number of fasteners, which may be configured to fasten onto the underside of the helmet portion 305. In certain embodiments, the suspension system 200 fasteners may be configured to secure and latch onto slots positioned within the opening under the helmet portion 305. The suspension system may include a tightening device 205, which may be configured to tighten or loosen the straps 210 of the suspension system 200 when the tightening device 205 is rotated left or right by the user. The straps 210 of the suspension system 200 may be configured to rest on the head of a user when the intelligent wearable device 300 is positioned on the head of the user.

The suspension system 200 may also include a processor 215, which may be secured to any portion of the suspension system 200, the helmet portion 305, and/or the bill portion 310. In certain embodiments, the processor 215 may configured to receive signals and sensor data from the other components of the intelligent wearable device 300, the other devices and networks of the system 100, or a combination thereof. The processor 215 may also be configured to process the signals, transmit the signals, generate outputs based on the signals, transmit the outputs, or any combination thereof. For example, the processor 215 may be configured to transmit signals via a radio integrated with the processor 215. In certain embodiments, the processor 215 may be configured to perform the operative functionality of the intelligent wearable device 300 and/or the functionality described for the method 400 described in the present disclosure. The processor 215 may also be integrated with a power source, such as a battery, which may provide power to the components of the intelligent wearable device 300. In certain embodiments, the power source may be configured to be rechargeable and may be configured to have at least 24-hour battery life. In certain embodiments, the suspension system 200 may also include any number of memories, which may be executed by the processor 215 to perform the various operations conducted by the intelligent wearable device 300.

In certain embodiments, the suspension system 200 may also include one or more light devices 217, which may be positioned on any portion of the suspension system 200, the helmet portion 305, the bill portion 310, and/or any other portion of the intelligent wearable device 300. The light devices 217 may be LEDs, optical lights, smart lights, any type of lights, or a combination thereof. In certain embodiments, the light devices 217 may be configured to output lights based on signals received from the processor 215, other components of the intelligent wearable device 300, and/or other devices of the system 100. For example, upon the detection of an event, the light devices 217 may turn from a default green color to a red color for a particular type of event and to a blue color upon the detection of another type of event. In certain embodiments, the light devices 217 may be configured to pulse in certain patterns and/or in random patterns depending on the signal received. For example, the light device 217 may pulse ten red flashes if the body temperature of the user has exceeded a threshold value, may pulse 5 blue flashes if the user needs to be relocated, and may pulse 2 green flashes if the action has been performed or if a detected event is no longer detected by the system 100.

The suspension system 200 may also include a sensor strip 220, which may be configured to be positioned on any portion of the suspension system 200. In a preferred embodiment, the sensor strip 200 may be positioned on the portion 222 of the suspension system 200 designed to rest on the forehead of the user when the intelligent wearable device 300 is positioned on the head of the user. The sensor strip 220 may be configured to be made of a flexible material and may include any number of sensors 225, 230, 235. In certain embodiments, additional sub-sensors may be embedded within the sensors 225, 230, 235 and may be interchanged depending on the type of sensors that are desired. The sensors 225, 230 235 may include, but are not limited to, temperature sensors, pressure sensors, motion sensors, light sensors, oxygen sensors, heart rate sensors, touch sensors, proximity sensors, gas sensors, acoustic sensors, chemical sensors, acceleration sensors, humidity sensors, moisture sensors, presence sensors, force sensors, any type of sensors, or a combination thereof. In certain embodiments, the sensor strip 220 and sensors 225, 230, 235 may be arranged in a mesh or other configuration (e.g. the mesh and/or other configurations as disclosed in U.S. Provisional Patent Application 62/953,309, filed on Dec. 24, 2019, which is hereby incorporated by reference in its entirety in this present disclosure), and may be configured to monitor various biological information and signals, health-related information and signals, environmental data and signals, and other information and signals, or a combination thereof. For example, the sensors 225, 230, 235 may be thermometers/thermistors that may be configured to measure the temperature on the forehead skin of a user. Notably, additional sensors 225, 230, 235 may also be positioned on the helmet portion 305, the bill portion 310, any other portion of the intelligent wearable device 300, on the body of the user, within the environment that the user is located in, and/or in any other location. The additional sensors 225, 230, 235 may be configured to measure environmental data, such as, but not limited to, the temperature in the environment, the pressure in the environment, whether motion is occurring in the environment, steam in the environment, gas in the environment, light in the environment, oxygen in the environment, liquid in the environment, moisture in the environment, sound in the environment, forces in the environment, anything in the environment, or a combination thereof.

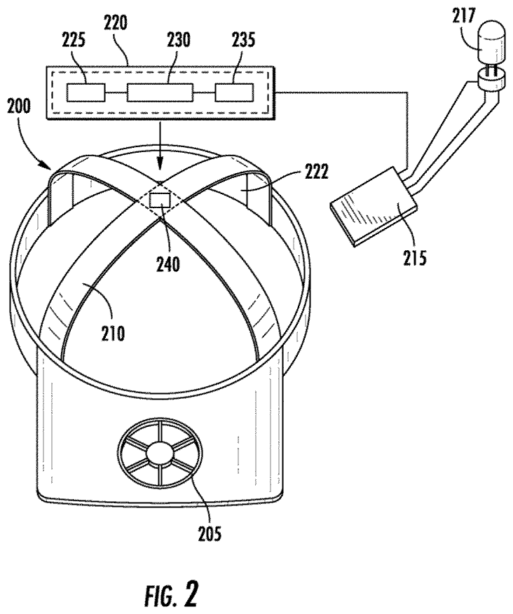

The suspension system 200 may also include another sensor 240, which may be similar to sensors 225, 230, 235. The sensor 240 may be positioned at the location where the straps 210 of the suspension system 200 overlap, as shown in FIG. 2. However, in certain embodiments, the sensor 240 may be positioned in any other desired location. In certain embodiments, the sensor 240 may be configured to measure the temperature underneath the helmet portion 305 of the intelligent wearable device 300 when not pressed against the user's skin. The temperature underneath the helmet portion may be normalized with the temperature measured on the forehead skin of the user and then mapped to the user's oral temperature (and/or rectal temperature and/or temperatures associated with other zones of the body depending on the situation), which may be measured by a thermometer or even another sensor 225, 230, 235, 240. Of course, the sensors 240 may also be pressure sensors, motion sensors, light sensors, oxygen sensors, heart rate sensors, touch sensors, proximity sensors, gas sensors, acoustic sensors, chemical sensors, acceleration sensors, humidity sensors, moisture sensors, presence sensors, force sensors, any type of sensors, or a combination thereof. Sensor 340 may be much like sensor 240 may be positioned on the outside portion of the intelligent wearable device 300 so as to measure sensor data occurring in the environment of the user.

Operatively and referring now also to FIGS. 1-3, the system 100 may operate according to the following exemplary use-case scenario. Notably, the system 100 is not limited to the specific use-case scenario described herein, and may be applied to any suitable and/or desired use-case scenario. In such a use-case scenario, a user, such as first user 101, may be located in a factory which may expose the first user 101 to various environmental and/or potentially unsafe conditions. When entering the factory, the first user 101 may secure the intelligent wearable device 300 to his head and may activate the intelligent wearable device 300 via a switch or button of the intelligent wearable device 300. Once activated, the sensors 225, 230, 235 may measure the forehead skin temperature of the first user 101, the sensor 240 can measure the temperature underneath the helmet portion 305, and the sensor 340 can measure the ambient temperature outside occurring in the environment. The components of the system 100 may be configured to map and/or normalize the forehead skin temperature relative to the temperature inside of the helmet portion 305 and compare this information to the oral temperature of the first user 101, which may be measured by another sensor. The sensors 225, 230, 235 may also be configured to measure oxygen saturation levels, heart rate, impact forces occurring on the first user 101, humidity levels associated with the first user 101 and/or environment, any other environmental and/or health data, or a combination thereof.

The intelligent wearable device 300 may analyze the measured temperature sensor data and compare the data to a threshold range of values or to a specific threshold value. For example, the threshold range of values may be 94 degrees to 99 degrees, or, in the event there is only one threshold value, the single threshold value may be 99 degrees. If measured temperature sensor data is 103 degrees and is thus outside the threshold range or beyond the single threshold value, the intelligent wearable device 300 may generate a signal indicating that the first user 101 is experiencing an event—in this case an abnormally high temperature. The intelligent wearable device 300 may also determine the type of event and one or more actions to perform in response to the event. In this scenario, the intelligent wearable device 300 may determine that the first user 101 is experiencing hyperthermia and may generate a signal that causes the light device 217, the interface 315, the speaker 325, the haptic component 330, the transceiver 335, and/or other components of the intelligent wearable device 300 to output an alert and/or notification indicative of the event and/or the type of the event. The signal may also indicate one or more actions to perform. In this scenario, the action to perform may be to have the first user 101 move himself out of the area or that another user, such as second user 110, move the first user 101 to another location—such as in the even that the first user 101 is incapacitated or weak. The signals may be transmitted to any of the devices in the system 100 including the intelligent wearable device 400 of the second user 110 and/or the second user device 111, who may be in a best position to assist the first user 101. Once the action is performed, the intelligent wearable device 300 may disable or remove any notifications and/or alerts and may continue to measure sensor data associated with the first user 101 and the environment. The process can repeat as new events are detected and responded to by the first user 101 and/or the second user 110.

Notably, as shown in FIG. 1, the system 100 may perform any of the operative functions disclosed herein by utilizing the processing capabilities of server 160, the storage capacity of the database 155, or any other component of the system 100 to perform the operative functions disclosed herein. The server 160 may include one or more processors 162 that may be configured to process any of the various functions of the system 100. The processors 162 may be software, hardware, or a combination of hardware and software. Additionally, the server 160 may also include a memory 161, which stores instructions that the processors 162 may execute to perform various operations of the system 100. For example, the server 160 may assist in processing loads handled by the various devices in the system 100, such as, but not limited to, activating and/or deactivating the intelligent wearable devices 300, 400; generating, receiving, and/or transmitting signals from the sensors of the intelligent wearable devices 300, 400; comparing health-related data, biological data, environmental data, and/or other data to a threshold range of values or to a threshold value associated with safety of a user (or other safety-related metric); determining if the comparing indicates that the health-related data, biological data, environmental data, and/or other data are outside the threshold range of values or threshold value; generating a signal indicating that a user is experiencing an event; outputting notifications providing information relating to the event and/or indicating one or more actions to perform; transmitting the generated signal to a different user and/or to a remote device to notify the different user and/or remote device of the event and/or one or more actions to perform with respect to the event; and performing any other suitable operations conducted in the system 100 or otherwise. In one embodiment, multiple servers 160 may be utilized to process the functions of the system 100. The server 160 and other devices in the system 100, may utilize the database 155 for storing data about the devices in the system 100 or any other information that is associated with the system 100. In one embodiment, multiple databases 155 may be utilized to store data in the system 100.

In certain embodiments, the system 100 may also include a computing device 170. The computing device 170 may include one or more processors 172 that may be configured to process any of the various functions of the system 100. The processors 172 may be software, hardware, or a combination of hardware and software. Additionally, the computing device 170 may also include a memory 171, which stores instructions that the processors 172 may execute to perform various operations of the system 100. For example, the computing device 170 may assist in processing loads handled by the various devices in the system 100, such as, but not limited to, the wearable devices 300, 400. In certain embodiments, for example, the intelligent wearable devices 300, 400 may offload certain types of less critical operations to the computing device 170 so that the intelligent wearable devices 300, 400 can focus on more critical operations.

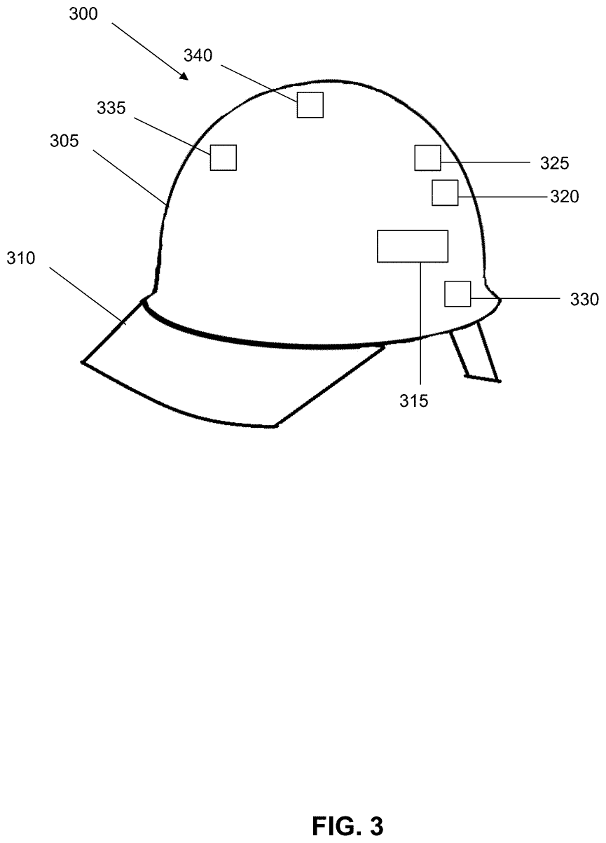

Although FIGS. 1-3 illustrates specific example configurations of the various components of the system 100, the system 100 may include any configuration of the components, which may include using a greater or lesser number of the components. For example, the system 100 is illustratively shown as including a first user device 102, a second user device 111, a database 125, a communications network 135, a server 140, a server 150, a server 160, a database 155, an external network 165, an intelligent wearable device 300, an intelligent wearable device 400, a suspension system 200, sensors 225, 230, 235, 240, processor 215, LED 217, tightening device 205, a sensor strip 220, an interface 315, a speaker 325, a microphone 320, a haptic component 330, and a transceiver 335. However, the system 100 may include multiple first user devices 102, multiple second user devices 111, multiple databases 125, multiple communications networks 135, multiple servers 140, multiple servers 150, multiple servers 160, multiple databases 155, multiple data warehouses 204, multiple intelligent wearable device 400, multiple intelligent wearable devices 400, multiple suspension systems 200, any number of sensors 225, 230, 235, 240, multiple processors, 215, multiple LEDs 217, multiple tightening devices 205, multiple sensor strips 220, multiple external networks 165, multiple interfaces 315, multiple speakers 325, multiple microphones 320, multiple haptic components 330, multiple transceivers 335, and/or any number of any of the other components inside or outside the system 100. Similarly, the system 100 may include any number of data sources, applications, systems, and/or programs. Notably, any of the components of the suspension system 200 as shown in FIG. 2 may be incorporated into the helmet portion 305 and/or the bill portion 310. Additionally, any of the components as shown in FIG. 3 may be integrated and/or positioned onto the suspension system 200 and/or the sensor strip 220. Furthermore, in certain embodiments, substantial portions of the functionality and operations of the system 100 may be performed by other networks and systems that may be connected to system 100.

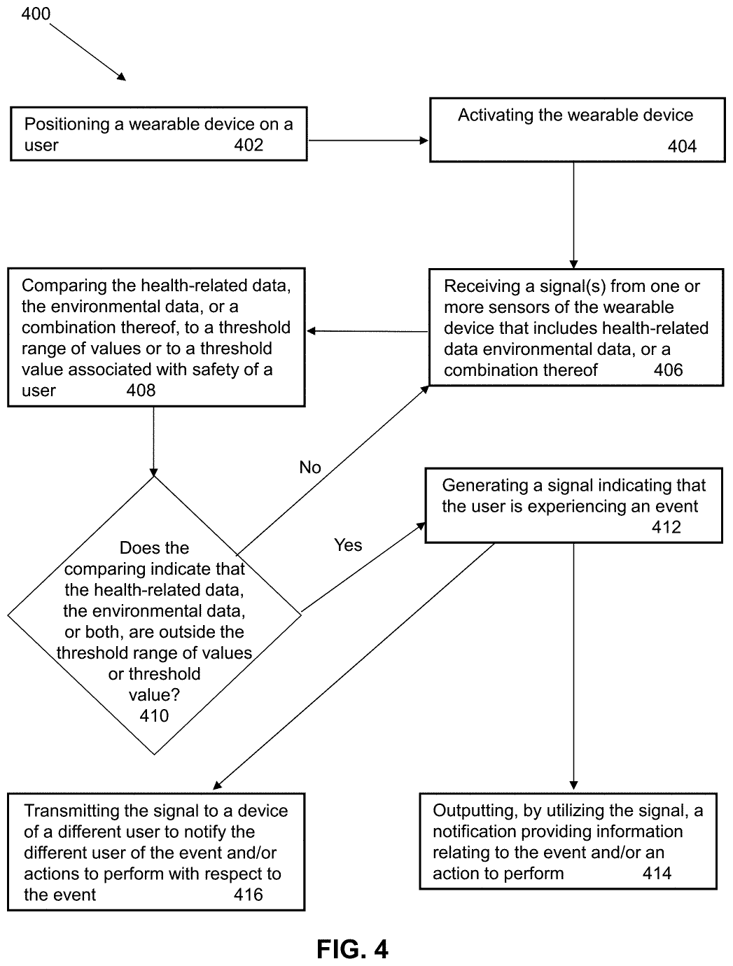

As shown in FIG. 4, an exemplary method 400 for providing and utilizing an intelligent wearable device, such as intelligent head protective equipment, is schematically illustrated. The method 400 may include, at step 402, positioning an intelligent wearable device (e.g. intelligent wearable device 300) on a user. For example, the intelligent wearable device 300 may be positioned on the head of the first user 101 or any other desired body part. In certain embodiments, the positioning of the intelligent wearable device may be performed by the first user 101 himself, another user, and/or even a robot or machine. For example, a robot and/or machine may determine a user's measurements and construct the intelligent wearable device 300 to be specifically fitted for the user. The robot and/or machine may include components, such as arms or projections, that may position and place the intelligent wearable device 300 on the appropriate body part of the user. The user may be located in an environment, such as a factory, ship, mine, and/or any other type of environment. Once the intelligent wearable device is positioned on the user, the method 400 may include, at step 404 activating the intelligent wearable device. In certain embodiments, the intelligent wearable device may be activated by the first user 101 pressing or flipping a power button of the intelligent wearable device, however, in certain embodiments, the intelligent wearable device may be activated by another device, such as the first user device 102 or even the second user device 111. In certain embodiments, the activating of the intelligent wearable device may be performed and/or facilitated by utilizing the first user device 102, the second user device 111, the server 140, the server 150, the server 160, the communications network 135, the external network 165, the database 155, the wearable device 300 the wearable device 400, the computing device 170, any appropriate program, device, network, and/or process of the system 100, or a combination thereof.

At step 406, the method 400 may include receiving one or more signals from one or more sensors of the intelligent wearable device. In certain embodiments, the sensors may be positioned anywhere on and/or within the intelligent wearable device itself. In certain embodiments, the sensors may be positioned on a suspension system (e.g. suspension system 200) of the intelligent wearable device. In a preferred embodiment, the sensors may be positioned on the portion of the suspension system that contacts the forehead of the user when the intelligent wearable device is worn on the head of the user. In certain embodiments, the signals may include measurement data measured and/or obtained by the sensors and may include environmental data, physiological data, biological data and/or health data associated with the user including, but not limited to, temperature data, oxygen saturation data, heart rate data, impact force data (or other force data), humidity data, pressure data, acceleration data, motion data, stability data (e.g. data that indicates if the user is wobbling or unstable), eye motion data, light data (e.g. ambient light data), moisture data, perspiration data, data indicative of detection of a chemical, gas, and/or substance, any type of environmental data, any type of health data, any type of biological data, any other data, or a combination thereof. In certain embodiments, the receiving of the one or more signals from the one or more sensors may be performed and/or facilitated by utilizing the first user device 102, the second user device 111, the server 140, the server 150, the server 160, the communications network 135, the external network 165, the database 155, the wearable device 300 the wearable device 400, the computing device 170, the sensors 225, 230, 235, 240, any appropriate program, device, network, and/or process of the system 100, or a combination thereof.

Once the one or more signals are received from the one or more sensors of the intelligent wearable device, the method 400 may include, at step 408, comparing the biological data, health data, environmental data, and/or other data in the one or more signals to a threshold range of values and/or to a threshold value. In certain embodiments, the threshold range of values or threshold value may be a threshold range of values or threshold value associated with the safety of the user. As an example, if the measured data indicates that the user's forehead skin temperature is 105 degrees Fahrenheit and the threshold range of values associated with safety is between 94 degrees Fahrenheit and 102 degrees Fahrenheit, the user's forehead skin temperature may be outside the threshold range of values associated with safety. As another example, if the measured data indicates that the user's forehead skin temperature is 105 degrees Fahrenheit, the user's oral temperature is at 98.6 degrees Fahrenheit, the temperature inside of the helmet portion of the intelligent wearable device is 103 degrees Fahrenheit, and the threshold temperature is 102 degrees Fahrenheit, the fact that the temperature inside the helmet and the forehead skin temperature are beyond 102 degrees may be detected by the intelligent wearable device by comparing the temperatures. As a further example, the forehead skin temperature (potentially along with skin temperatures from other areas of skin) may be compared and normalized to the temperature inside of the helmet portion of the intelligent wearable device, and the correlation between the temperature inside of the helmet portion and the forehead skin temperature may be mapped to the oral temperature of the wearer. Based on the foregoing information, the system 100 may determine that the normalized temperature inside of the helmet portion and forehead skin temperature deviate from the user's oral temperature by a threshold value. In certain embodiments, instead of monitoring temperature, any other environmental, biological, health-related, and/or other signals may be monitored as well, such as, but not limited to, oxygen heart rate, impact, force, and/or humidity. In certain embodiments, the comparing may be performed and/or facilitated by utilizing the first user device 102, the second user device 111, the server 140, the server 150, the server 160, the communications network 135, the external network 165, the database 155, the wearable device 300 the wearable device 400, the computing device 170, the sensors 225, 230, 235, 240, any appropriate program, device, network, and/or process of the system 100, or a combination thereof.

At step 410, the method 400 may include determining if the comparing indicates that the biological data, health data, environmental data, and/or other data present in the one or more signals is outside the threshold range of values and/or beyond a threshold value. In certain embodiments, the determining may be performed and/or facilitated by utilizing the first user device 102, the second user device 111, the server 140, the server 150, the server 160, the communications network 135, the external network 165, the database 155, the wearable device 300 the wearable device 400, the computing device 170, the sensors 225, 230, 235, 240, any appropriate program, device, network, and/or process of the system 100, or a combination thereof. If, at step 410, the determining indicates that the biological data, health data, environmental data, and/or other data present in the one or more signals is not outside the threshold range of values and/or not beyond the threshold value, the method 400 may revert back to step 406 and continue to receive signals from the sensors of the intelligent wearable device until data is outside the threshold range of values and/or beyond the threshold value. If, however, at step 410, the determining indicates that the biological data, health data, environmental data, and/or other data present in the one or more signals is outside the threshold range of values and/or beyond the threshold value, the method 400 may proceed to step 412, which may include generating a signal indicating that the user is experience an event. In certain embodiments, the generating of the signal may be performed and/or facilitated by utilizing the first user device 102, the second user device 111, the server 140, the server 150, the server 160, the communications network 135, the external network 165, the database 155, the wearable device 300 the wearable device 400, the computing device 170, the sensors 225, 230, 235, 240, any appropriate program, device, network, and/or process of the system 100, or a combination thereof.

An event may be anything that the user is experiencing, anything that is occurring in the environment in which the user is located, any interaction between the user and the environment, an interaction between the user and another user, an interaction between the user and another living thing, an interaction between the user and an object, any type of event, or a combination thereof. For example, the event may be that the user's forehead temperature and/or the temperature within the helmet portion of the intelligent wearable device has increased beyond a threshold value associated with the user's safety. In such a scenario, if the user's forehead skin temperature is 102 degrees and the threshold value for safety is 100 degrees, the signal generated at step 412 may include an identification of the temperature and an identification that the temperature is beyond the threshold. In certain embodiments, the signal may include any other information including, but not limited to, any of the data obtained from the sensors. Once the signal indicating that the user is experiencing an event is generated, the method 400 may include, at step 414, outputting a notification providing information relating to the event and/or an action to perform, such as in response to the event. For example, a light (e.g. LED) of the intelligent wearable device may change from green to red to notify that an emergency event is occurring. As another example, a speaker of the intelligent wearable device may output a specific sound and/or sequence of sounds upon detection of the event. As a further example, a user interface of the intelligent wearable device may render a visual alert that may be visible by the user or by other users in proximity to the user wearing the intelligent wearable device. In further embodiments, any type of notification may be provided including, but not limited to, a phone call, a text message, an instant message, a haptic output (e.g. certain type of vibrations indicative of certain types of events), a visual output, an auditory output, a chemical output, a video output, a radio output, any type of output, or a combination thereof. In certain embodiments, the notification may be provided and/or facilitated by utilizing the first user device 102, the second user device 111, the server 140, the server 150, the server 160, the communications network 135, the external network 165, the database 155, the wearable device 300 the wearable device 400, the computing device 170, the sensors 225, 230, 235, 240, any appropriate program, device, network, and/or process of the system 100, or a combination thereof.

In certain embodiments, the notification outputted based on the signal may also include identify one or more actions to perform with respect to the event. For example, if the event is that the user is overheating, the action to perform may include moving the user to a new location and applying ice to the user's face and/or body in a certain fashion. As another example, if the event that the user is experiencing is that the user is inhaling too much carbon monoxide or some other gas or chemical, the action to perform may be to take the user to a hospital and to administer a specific medical treatment and/or medication to counteract the effects of the gas or chemical. At step 416, the method 400 may include transmitting the signal generated at step 412 and/or a notification based on the signal to a different user to notify the different user of the event and/or to actions to perform with respect to the detected event. In certain embodiments, the transmitting of the signal and/or notification may be performed and/or facilitated by utilizing the first user device 102, the second user device 111, the server 140, the server 150, the server 160, the communications network 135, the external network 165, the database 155, the wearable device 300 the wearable device 400, the computing device 170, the sensors 225, 230, 235, 240, any appropriate program, device, network, and/or process of the system 100, or a combination thereof. Once the signals and/or notifications are provided, the user himself may perform one or more actions in response to the event and/or other users may perform one or more actions in response to the event. Notably, the method 400 may further incorporate any of the features and functionality described for the system 100 or as otherwise described herein.

The systems and methods disclosed herein may include additional functionality and features. For example, the operative functions of the system 100 and method may be configured to execute on a special-purpose processor specifically configured to carry out the operations provided by the system 100 and method. Notably, the operative features and functionality provided by the system 100 and method may increase the efficiency of computing devices that are being utilized to facilitate the functionality provided by the system 100 and method 400. For example, through the use of the artificial intelligence and machine learning in conjunction with the system 100 and/or method 400, a reduced amount of computer operations need to be performed by the devices in the system 100 using the processors and memories of the system 100 than in systems that are not capable of machine learning as described in this disclosure. As an illustration and in certain embodiments, the system 100 may learn over time that certain environmental and/or biological data are associated with certain health conditions. For example, if the system 100 initially determines during a first occasion that measured biological data of the user and/or environmental data is outside a threshold range of values associated with the safety, the system 100 may determine a health condition that is occurring for the user. Knowing this information, the system 100 may automatically determine the health condition for the user during a future second occasion if newly measured biological and/or environmental data matches the biological data and/or environmental data from the first occasion. As a result, in such a scenario, the system 100 would not have to compare the newly measured biological and/or environmental data to the threshold range of values because the system 100 already has determined that the health condition would exist based on the measured data. In such a context, less processing power needs to be utilized because the processors and memories do not need perform analyses and operations that have already been learned by the system 100. As a result, there are substantial savings in the usage of computer resources by utilizing the software, functionality, and algorithms provided in the present disclosure.

Notably, in certain embodiments, various functions and features of the system 100 and methods may operate without human intervention and may be conducted entirely by computing devices, robots, and/or processes. For example, in certain embodiments, multiple computing devices may interact with devices of the system 100 to provide the functionality supported by the system 100. Additionally, in certain embodiments, the computing devices of the system 100 may operate continuously to reduce the possibility of errors being introduced into the system 100. In certain embodiments, the system 100 and methods may also provide effective computing resource management by utilizing the features and functions described in the present disclosure. For example, in certain embodiments, while comparing measured biological or health data and environmental data to a threshold range of values, any selected device in the system 100 may transmit a signal to a computing device receiving or processing the input that only a specific quantity of computer processor resources (e.g. processor clock cycles, processor speed, processor cache, etc.) may be dedicated to processing the data during the comparison stage, processing any other operation conducted by the system 100, or any combination thereof. For example, the signal may indicate an amount of processor cycles of a processor that may be utilized to process the data during the comparison stage, and/or specify a selected amount of processing power that may be dedicated to processing the data and/or any of the operations performed by the system 100. In certain embodiments, a signal indicating the specific amount of computer processor resources or computer memory resources to be utilized for performing an operation of the system 100 may be transmitted from the first and/or second user devices 102, 111 and/or intelligent wearable devices 300, 400 to the various components and devices of the system 100.

In certain embodiments, any device in the system 100 may transmit a signal to a memory device to cause the memory device to only dedicate a selected amount of memory resources to the various operations of the system 100. In certain embodiments, the system 100 and methods may also include transmitting signals to processors and memories to only perform the operative functions of the system 100 and methods at time periods when usage of processing resources and/or memory resources in the system 100 is at a selected, predetermined, and/or threshold value. In certain embodiments, the system 100 and methods may include transmitting signals to the memory devices utilized in the system 100, which indicate which specific portions (e.g. memory sectors, etc.) of the memory should be utilized to store any of the data utilized or generated by the system 100. Notably, the signals transmitted to the processors and memories may be utilized to optimize the usage of computing resources while executing the operations conducted by the system 100. As a result, such features provide substantial operational efficiencies and improvements over existing technologies.

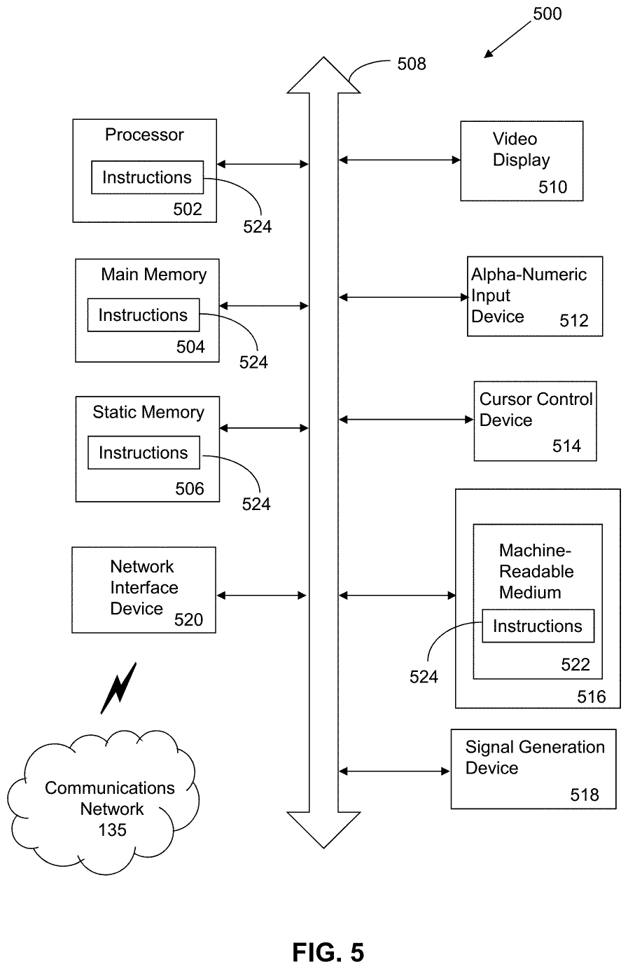

Referring now also to FIG. 5, at least a portion of the methodologies and techniques described with respect to the exemplary embodiments of the system 100 can incorporate a machine, such as, but not limited to, computer system 500, or other computing device within which a set of instructions, when executed, may cause the machine to perform any one or more of the methodologies or functions discussed above. The machine may be configured to facilitate various operations conducted by the system 100. For example, the machine may be configured to, but is not limited to, assist the system 100 by providing processing power to assist with processing loads experienced in the system 100, by providing storage capacity for storing instructions or data traversing the system 100, or by assisting with any other operations conducted by or within the system 100.

In some embodiments, the machine may operate as a standalone device. In some embodiments, the machine may be connected (e.g., using communications network 135, another network, or a combination thereof) to and assist with operations performed by other machines, programs, functions, and systems, such as, but not limited to, the first user device 102, the second user device 111, the server 140, the server 150, the database 155, the server 160, the intelligent wearable devices 300, 400, the suspension system 200, the sensors 225, 230, 235, the processor 215, the external network 165, the communications network 135, any device, system, and/or program in FIGS. 1-5, or any combination thereof. The machine may be connected with any component in the system 100. In a networked deployment, the machine may operate in the capacity of a server or a client user machine in a server-client user network environment, or as a peer machine in a peer-to-peer (or distributed) network environment. The machine may comprise a server computer, a client user computer, a personal computer (PC), a tablet PC, a laptop computer, a desktop computer, a control system, a network router, switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The computer system 500 may include a processor 502 (e.g., a central processing unit (CPU), a graphics processing unit (GPU, or both), a main memory 504 and a static memory 506, which communicate with each other via a bus 508. The computer system 500 may further include a video display unit 510, which may be, but is not limited to, a liquid crystal display (LCD), a flat panel, a solid state display, or a cathode ray tube (CRT). The computer system 500 may include an input device 512, such as, but not limited to, a keyboard, a cursor control device 514, such as, but not limited to, a mouse, a disk drive unit 516, a signal generation device 518, such as, but not limited to, a speaker or remote control, and a network interface device 520.

The disk drive unit 516 may include a machine-readable medium 522 on which is stored one or more sets of instructions 524, such as, but not limited to, software embodying any one or more of the methodologies or functions described herein, including those methods illustrated above. The instructions 524 may also reside, completely or at least partially, within the main memory 504, the static memory 506, or within the processor 502, or a combination thereof, during execution thereof by the computer system 500. The main memory 504 and the processor 502 also may constitute machine-readable media.

Dedicated hardware implementations including, but not limited to, application specific integrated circuits, programmable logic arrays and other hardware devices can likewise be constructed to implement the methods described herein. Applications that may include the apparatus and systems of various embodiments broadly include a variety of electronic and computer systems. Some embodiments implement functions in two or more specific interconnected hardware modules or devices with related control and data signals communicated between and through the modules, or as portions of an application-specific integrated circuit. Thus, the example system is applicable to software, firmware, and hardware implementations.

In accordance with various embodiments of the present disclosure, the methods described herein are intended for operation as software programs running on a computer processor. Furthermore, software implementations can include, but not limited to, distributed processing or component/object distributed processing, parallel processing, or virtual machine processing can also be constructed to implement the methods described herein.

The present disclosure contemplates a machine-readable medium 522 containing instructions 524 so that a device connected to the communications network 135, the external network 165, another network, or a combination thereof, can send or receive voice, video or data, and communicate over the communications network 135, the external network 165, another network, or a combination thereof, using the instructions. The instructions 524 may further be transmitted or received over the communications network 135, the external network 165, another network, or a combination thereof, via the network interface device 520.

While the machine-readable medium 522 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that causes the machine to perform any one or more of the methodologies of the present disclosure.

The terms “machine-readable medium,” “machine-readable device,” or “computer-readable device” shall accordingly be taken to include, but not be limited to: memory devices, solid-state memories such as a memory card or other package that houses one or more read-only (non-volatile) memories, random access memories, or other re-writable (volatile) memories; magneto-optical or optical medium such as a disk or tape; or other self-contained information archive or set of archives is considered a distribution medium equivalent to a tangible storage medium. The “machine-readable medium,” “machine-readable device,” or “computer-readable device” may be non-transitory, and, in certain embodiments, may not include a wave or signal per se. Accordingly, the disclosure is considered to include any one or more of a machine-readable medium or a distribution medium, as listed herein and including art-recognized equivalents and successor media, in which the software implementations herein are stored.

The illustrations of arrangements described herein are intended to provide a general understanding of the structure of various embodiments, and they are not intended to serve as a complete description of all the elements and features of apparatus and systems that might make use of the structures described herein. Other arrangements may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Thus, although specific arrangements have been illustrated and described herein, it should be appreciated that any arrangement calculated to achieve the same purpose may be substituted for the specific arrangement shown. This disclosure is intended to cover any and all adaptations or variations of various embodiments and arrangements of the invention. Combinations of the above arrangements, and other arrangements not specifically described herein, will be apparent to those of skill in the art upon reviewing the above description. Therefore, it is intended that the disclosure not be limited to the particular arrangement(s) disclosed as the best mode contemplated for carrying out this invention, but that the invention will include all embodiments and arrangements falling within the scope of the appended claims.

The foregoing is provided for purposes of illustrating, explaining, and describing embodiments of this invention. Modifications and adaptations to these embodiments will be apparent to those skilled in the art and may be made without departing from the scope or spirit of this invention. Upon reviewing the aforementioned embodiments, it would be evident to an artisan with ordinary skill in the art that said embodiments can be modified, reduced, or enhanced without departing from the scope and spirit of the claims described below.

We claim:

1. A wearable device, comprising:
a helmet;
a sensor coupled to the helmet; and
a processor that executes instructions to perform operations, the operations comprising:
receiving a first signal from the sensor, wherein the first signal includes health-related data associated with a first user of the wearable device and environmental data associated with an environment associated with the first user;
comparing the health-related data associated with the first user, the environmental data, or a combination thereof, to a threshold value associated with safety of the first user;
generating, if the comparing indicates that the health-related data, the environmental data, or both, are outside the threshold value, a second signal indicating that the first user is experiencing an event.

2. The wearable device of claim 1, wherein the operations further comprise transmitting the second signal to a device of a second user, wherein the operations further comprise facilitating output of a notification on the device of the second user that indicates and action to be performed with respect to the first user.

3. The wearable device of claim 1, wherein the health-related data comprises temperature data, oxygen saturation data, heart rate data, impact force data, humidity data, sweat-related data, breathing-related data, glucose-related data, blood-related data, biometric data, fatigue-related data, any health-related data, or a combination thereof.

4. The wearable device of claim 1, wherein the operations further comprise utilizing the second signal to output a visual notification on a user interface of the wearable device, an audible alert via a speaker of the wearable device, activate a light of the wearable device, haptic feedback via a haptic component of the wearable device, any type of alert, or a combination thereof.

5. The wearable device of claim 1, wherein the operations further comprise utilizing the second signal to output a notification indicating that the user needs to relocation to a new location.

6. The wearable device of claim 1, wherein the operations further comprise monitoring the first signal and subsequent signals received from the sensor over time.

7. The wearable device of claim 6, wherein the operations further comprise determining a correlation between the environmental data and a biological change of the user based on the first signal and the subsequent signals.

8. The wearable device of claim 1, wherein the operations further comprise prompting the user for feedback relating to the event after generating the signal.

9. The wearable device of claim 8, wherein the operations further comprise receiving the feedback from the user, and wherein the operations further comprise performing an action in response to the feedback.

10. The wearable device of claim 1, wherein the sensor comprises a plurality of sensors, and wherein the plurality of sensors comprise a primary sensor and a sub-sensor implemented directly into the primary sensor.

11. The wearable device of claim 1, wherein the operations further comprise providing the second signal to a radio of the wearable device, and wherein the operations further comprise causing the radio to transmit the second signal to another device so as to obtain assistance for the user experiencing the event

12. The wearable device of claim 1, wherein the operations further comprise determining a health condition associated with the user based on the event and based on the comparing.

13. The wearable device of claim 1, wherein the operations further comprise causing the second signal to be transmitted for output on a headphone of the wearable device, a headphone of another device, or a combination thereof.

14. A method, comprising:
obtaining a first signal from a sensor of a wearable device, wherein the first signal includes health-related data associated with a first user of the wearable device and environmental data associated with an environment associated with the first user;
comparing, by utilizing instructions executed by a processor of the wearable device, the health-related data associated with the first user, the environmental data, or a combination thereof, to a threshold value associated with safety of the first user; and
providing, if the comparing indicates that the health-related data, the environmental data, or both, are outside the threshold value, a second signal indicating that the first user is experiencing an event.

15. The method of claim 14, wherein the health-related data comprises first temperature sensor data obtained from skin of the first user, second temperature sensor data inside a helmet of the wearable device, and third temperature sensor data from an oral temperature of the first user.

16. The method of claim 15, further comprising mapping the first temperature sensor data to the second temperature sensor data and comparing the mapping to the third temperature sensor data from the oral temperature to determine if the health-related data is outside the threshold value.

17. The method of claim 14, wherein the sensor comprises a plurality of sensors, and further comprising positioning a first sensor of the plurality of sensors on a forehead of the first user, further comprising positioning a second sensor of the plurality of sensors on a suspension system of the wearable device, and further comprising positioning a third sensor of the plurality of sensors on the user at a location outside of the wearable device.

18. The method of claim 14, further comprising activating, based on the second signal, a haptic component of the wearable device, a light of the wearable device, a speaker of the wearable device, a user interface of the wearable device, any component of the wearable device, or a combination thereof.

19. The method of claim 14, further comprising utilizing the second signal to prompt an action to be performed with respect to the event experienced by the first user.

20. A non-transitory computer-readable device comprising instructions, which when loaded and executed by a processor, cause the processor to perform operations comprising:
receiving a first signal from a sensor of a wearable device, wherein the first signal includes health-related data associated with a first user of the wearable device and environmental data associated with an environment associated with the first user;
comparing, by utilizing instructions executed by a processor of the wearable device, the health-related data associated with the first user, the environmental data, or a combination thereof, to a threshold value associated with safety of the first user; and
determining, if the comparing indicates that the health-related data, the environmental data, or both, are outside the threshold value, that the first user is experiencing an event.
